(19) United States
(12) Patent Application Publication
(10) Pub. No.: US 2017/0027542 A1
(43) Pub. Date: Feb. 2, 2017

(54) MEDICAL IMAGING APPARATUS

(71) Applicant: SAMSUNG MEDISON CO., LTD., Hongcheon-gun (KR)

(72) Inventors: Kyeong Gu WOO, Suwon-si (KR); So Ra YANG, Goyang-si (KR); Sim Gwan JEONG, Seoul (KR); Jae Moon JO, Seongnam-si (KR); Seong Jin CHUNG, Seongnam-si (KR)

(21) Appl. No.: 15/183,683

(22) Filed: Jun. 15, 2016

(30) Foreign Application Priority Data
Jul. 30, 2015 (KR) 10-2015-0107718

(51) Int. Cl. A61B 8/00; A61B 8/08; G10K 11/178

(52) U.S. Cl. CPC A61B 8/4411; G10K 11/178; A61B 8/546; A61B 8/5207; A61B 8/461; A61B 8/467; A61B 8/4405; G10K 2210/116

(57) ABSTRACT

Disclosed is a medical imaging apparatus including a body and a noise reducing apparatus that is detachably disposed in the body to reduce noise generated from the body, wherein the noise reducing apparatus includes a neck and a cavity. The noise reducing apparatus disposes the neck and the cavity that are combined with each other according to a frequency of the noise generated from the medical imaging apparatus, thereby reducing noise generated from the medical imaging apparatus in an active manner, achieving improved silence.

### CROSS-REFERENCE TO RELATED APPLICATION

This application claims the benefit of Korean Patent Application No. 10-2015-0107718, filed on Jul. 30, 2015, in the Korean Intellectual Property Office, the disclosure of which is incorporated herein by reference.

### BACKGROUND

1. Field

Embodiments of the present disclosure relate to a medical imaging apparatus including a noise reducing apparatus.

2. Description of the Related Art

A medical imaging apparatus is an apparatus configured to obtain an internal image of a human body by allowing a constant energy to pass through a human body and using characteristics of incidence, reflection or transmission depending on the density and property of internal parts of the human body. For decades, the medical imaging was achieved only by applications of radioactive rays, but in the recent years, various medical imaging devices have been developed using ultrasonic waves, magnetic field, and laser.

A ultrasound imaging apparatus, an example of the medical imaging apparatus, obtains an image of the inside of an object, such as a soft tissue tomogram or a blood stream tomogram in a non-invasive manner by emitting ultrasound signals toward a target region from the surface of the object and using reflected ultrasound signals (ultrasound eco signals).

The ultrasound imaging apparatus has a compact structure, a low cost, a real-time operation and no risk of exposure to radioactive rays, as compared to other diagnostic imaging apparatuses, such as X-ray diagnosis apparatuses, X-ray Computerized Tomography Scanner (CT scanner), magnetic resonance imaging (MRI) apparatuses, and nuclear medicine diagnosis apparatuses. Therefore, the ultrasound imaging apparatus is widely used for diagnoses of the heart, the abdomen, urinary and maternity diagnoses.

### SUMMARY

Therefore, it is an aspect of the present disclosure to provide a medical imaging apparatus including a noise reducing apparatus for reducing flow noise of a fan mounted inside a body of the medical imaging apparatus.

Additional aspects of the invention will be set forth in part in the description which follows and, in part, will be obvious from the description, or may be learned by practice of the invention.

In accordance with an embodiment of the present disclosure, a medical imaging apparatus includes a body and a noise reducing apparatus. The noise reducing apparatus may be disposed inside the body to reduce noise generated from the body. The noise reducing apparatus may include a neck and a cavity, and the neck may be combined with the cavity according to a frequency of the noise desired to be reduced.

The body may further include a cooling apparatus to cool heat inside of the body, and the noise reducing apparatus may reduce noise generated from the cooling apparatus.

The medical imaging apparatus may further include: a processing unit disposed inside the body to process a medical image and a housing disposed inside the body to accommodate the processing unit. The noise reducing apparatus may be mounted at the housing.

The housing may include a mounting part on which the noise reducing apparatus is mounted, and the neck of the noise reducing apparatus may be detachable coupled to the mounting part and the cavity may be detachably coupled to the neck.

The neck may include a first portion connected to the mounting part and a second portion to which the cavity is connected. The first portion or the second portion may be provided in the form of a cylinder or a square pillar.

The cavity may be provided in the form of a cylinder or a hexahedral box that has an inside thereof empty, and the cavity may be provided at one side thereof with a connection part connected to the neck in the form of a hollow cylinder or a hollow square pillar.

The neck may include a screw thread formed on an outer surface of the first portion so as to be coupled to the mounting part and a screw thread formed on an inner surface of the second portion so as to be coupled to the cavity, and the cavity may include a screw thread formed on an outer surface of the connection part so as to be coupled to the second portion of the neck.

Each of the neck and the cavity of the noise reducing apparatus may be provided in plural, outer diameters of the first portions of the plurality of necks or cross-sections of the first portions including outer walls of the first portions of the plurality of necks may have uniform sizes and shapes among each other, and inner diameters of the first portions of the plurality of necks or cross-sections of hollow portions of the plurality of necks except for outer walls of the first portions of the plurality of necks may have different sizes and shapes from each other.

Outer diameters of the connection parts of the plurality of cavities or cross-sections of the connection parts of the plurality of cavities including outer walls of the connection parts of the plurality of cavities may have uniform sizes and shapes among each other, and inner volumes of the plurality of cavities may be different from each other.

The mounting part may be formed on one surface of the housing. The mounting part may include: an opening allowing the inside of the housing to communicate with the noise reducing apparatus and a sidewall surrounding the opening and to which the neck of the noise reducing apparatus is connected.

A screw thread may be formed on an inner surface of the sidewall of the mounting part so as to be coupled to the neck.

The housing may include a cooling apparatus to cool heat inside of the housing generated from the processing unit, and the noise reducing apparatus may reduce noise generated from the cooling apparatus.

The body further includes: a platform configured to support the housing at a lower side of the housing; and a cover configured to encase the housing to form an external appearance of the body, wherein the platform or the cover may be provided with a suction portion allowing outside air to be introduced into the body.

The cooling apparatus may be provided as a fan that discharges air suctioned through the suction port, and the noise reducing apparatus may be disposed on a flow path along which air introduced through the suction port moves to the fan.

The cooling apparatus may be mounted on a rear surface of the housing, and the noise reducing apparatus may be mounted on a upper surface or a side surface of the housing.

The cover may include an exhaust port provided at a position corresponding to the cooling apparatus that is disposed on the rear surface of the housing.

The processing unit may be accommodated in a case, and the case may include a separate fan and a separate noise reducing apparatus, and the case may be disposed inside the housing.

In accordance with an embodiment of the present disclosure, a medical imaging apparatus includes: a detection unit configured to acquire a detection signal and transmit the acquired detection signal; a body including a processing unit that is configured to convert the detection signal transmitted from the detection unit into an image signal; and an output unit configured to receive the image signal from the processing unit and display the received image signal, wherein the body may include a noise reducing apparatus configured to reduce noise generated from the body.

The body may further include a cooling apparatus to cool heat inside of the body, and the noise reducing apparatus may reduce noise generated from the cooling apparatus

The noise reducing apparatus may be detachably mounted inside of the body so as to be replaced according to a frequency of noise desired to be reduced.

The noise reducing apparatus may include a neck and a cavity, wherein the neck may be detachably coupled to the body and the cavity may be detachably coupled to the neck, so that the neck is combined with the cavity according to a frequency of the noise desired to be reduced.

The body may include a suction portion allowing outside air to be introduced into the body, and the noise reducing apparatus may be disposed on a flow path along which air introduced through the suction port moves to the cooling apparatus.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a front perspective view illustrating a medical imaging apparatus in accordance with an embodiment of the present disclosure;

FIG. 2 is an exploded perspective view illustrating the medical imaging apparatus of FIG. 1 with a body separated from a cover;

FIG. 3 is a bottom perspective view illustrating the body with the cover shown in FIG. 2 separated therefrom;

FIG. 4 is a rear perspective view illustrating the body with the cover shown in FIG. 2 separated therefrom;

FIG. 5 is a perspective view illustrating a processing unit case separated from the body shown in FIG. 4;

FIG. 6 is an exploded and enlarged perspective illustrating a noise reducing apparatus shown in FIG. 4;

FIG. 7 is a cross sectional view illustrating the noise reducing apparatus shown in FIG. 4;

FIG. 8 is an exploded cross sectional view illustrating the noise reducing apparatus shown in FIG. 7;

FIG. 9 is a cross sectional view illustrating the noise reducing apparatus shown in FIG. 4 in accordance with another embodiment of the present disclosure; and

FIG. 10 is an exploded cross sectional view illustrating the noise reducing apparatus of FIG. 9.

### DETAILED DESCRIPTION

Hereinafter, a medical imaging apparatus according to an embodiment of the present disclosure will be described with reference to the accompanying drawings, in which a ultrasonic medical imaging apparatus will be described as an example of the medical imaging apparatus.

The medical imaging apparatus according to an embodiment of the present disclosure includes a body, a detection unit connected to the body to acquire and transmit a signal detecting inside of a human body, a processing unit disposed in the body and configured to convert the detection signal received from the detection unit into an image signal, an output unit configured to receive the converted image signal from the processing unit and display the received signal, and a control unit configured to control each of the detection unit, the processing unit and the output unit.

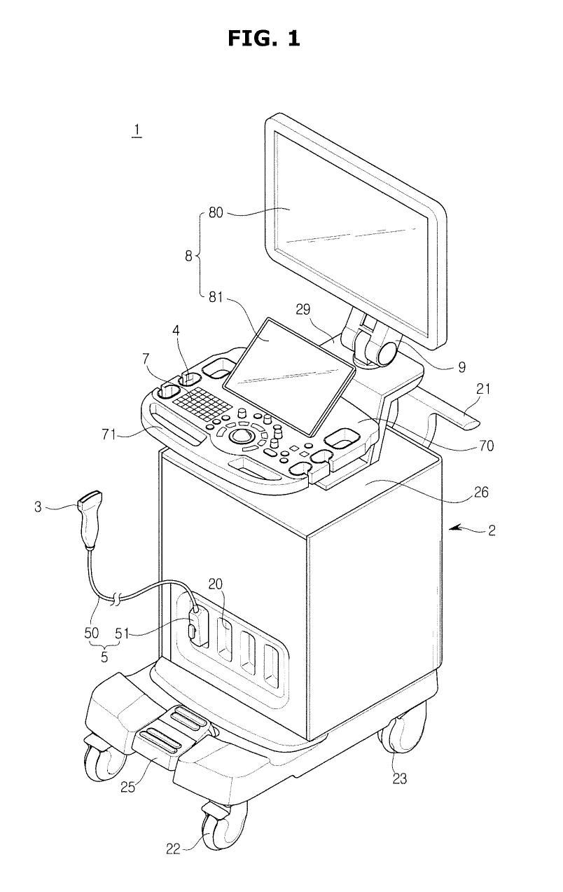

FIG. 1 is a front perspective view illustrating a medical imaging apparatus in accordance with an embodiment of the present disclosure.

Referring to FIG. 1, the ultrasonic imaging apparatus 1 includes a body 2, a ultrasonic probe 3 serving as a detection unit, an input unit 7 serving as a control unit, and a display member 8 serving as an output unit. The display member 8 may include a main display 80 and a sub-display 81.

The display member 8 may display a ultrasonic image obtained in a ultrasonic diagnosis process. In addition, the display member 8 may display an application associated with an operation of the ultrasonic imaging apparatus 1. For example, the main display 80 may display an ultrasonic image obtained in a ultrasonic diagnosis process. The sub-display 81 may display details associated with an operation of the ultrasonic imaging apparatus 1.

The main display 80 or the sub-display 81 may be implemented as a cathode ray tube (CRT), a liquid crystal display (LCD). The main display 80 or the sub-display 81 may be coupled to the body 2, or may be separated from the body 2.

The ultrasonic imaging apparatus 1 may further include a moving unit 9. The moving unit 9 may connect the body 2 to the display member 8. The moving unit 9 may be configured to allow the display member 8 to be movable with respect to the body 2. The moving unit 9 allows the display member 8 to be disposed while spaced apart from the body 2.

The body 2 may be provided with an input unit 7. The input unit 7 may be provided in the form of as a keyboard, a button, a dial, a foot switch or a foot pedal. The input unit 7 provided in the form of a keyboard may be provided at an upper side of the body 2. The input unit 7 provided in the form of a foot switch or a foot pedal may be provided at a lower side of the body 2. A test operator may control an operation of the ultrasonic imaging apparatus 1 through the input unit 7.

The input unit 7 may include an input panel 70 and a key board, a button and a dial provided on the input panel 70. The input panel 70 may be mounted on the body 2. At one side of the input panel 70, a handle part 71 may be provided. A user may move the ultrasonic imaging apparatus 1 by grasping the handle part 71 and applying a force to the handle part 71.

The ultrasonic probe 3 may be connected to the body 2 by a connection member 5. The connection member 5 includes a cable 50 and a connector 51. The ultrasonic probe 3 may be provided at one side of the cable 50 and the connector 51 may be provided at the other side of the cable 50. The connector 51 may be detachably mounted at a connecting part 20 provided at the body 2, so that the ultrasonic probe 3 is connected to the body 2.

At one side of the ultrasonic imaging apparatus 1, a mount 4 is provided such that the ultrasonic probe 3 is supported on the body 2. When the operator does not use the ultrasonic imaging apparatus 1, the ultrasonic probe 3 may be supported by the mount 4 and kept. For example, the mount 4 may be provided in the form of a hole provided in the input panel 70, allowing a gripping part of the ultrasonic probe 3 to pass therethrough. The ultrasonic probe 3 may be inserted into the hole formed through the input panel 70, and supported. For example, the mount 4 may be provided in the form of a holder mounted at the body 2. The ultrasonic probe 3 may be inserted into the holder and supported.

The body 2 is provided at a front side and a rear side thereof with a handle part 21 and a handle part 71, respectively, to move the ultrasonic imaging apparatus 1. The handle parts 21 and 71 may include a first handle part 21 provided at the front side of the body 2 and a second handle part 71 provided at the rear side of the body 2. The first handle part 71 may be provided at one side of the input panel 7.

The second handle part 21 may be provided at a rear side of the body 2 in a protruding manner.

The body 2 is provided with a plurality of casters 22 and 23 to move the ultrasonic imaging apparatus 1. The casters 22 and 23 may be arranged to travel the body 2 in a certain direction (in an arrangement travel mode) or may be provided to move freely (in a free travel mode), or may be locked to stop at a certain position of the body 2 (in a stop mode).

The casters 22 and 23 may include a first caster 22 and a second caster 23. When a side of the input part 7 and the display member 8 is referred to a front and the opposite side is referred to as a rear, the first caster 22 may be positioned in front of the body 2 and the second caster 34 may be positioned in rear of the body 2. The first caster 22 may be provided at both sides of the front of the body 2. The second caster 23 may be provided at both sides of the rear of the body 2 in correspondence to the first caster 22.

The body 2 may be provided with a manipulation part 25 to control the casters 22 and 23. The manipulation part 25 may be provided in the form of a foot pedal shown in FIG. 1, and may be provided in the form of a button and a dial. A user, after stepping on the foot pedal 25 for manipulation, may move or stop the ultrasonic imaging apparatus 1 while grasping the first handle part 71.

The following description will be made in relation to the body 2 of the medical imaging apparatus on which a noise reducing apparatus according to an embodiment of the present disclose is disposed.

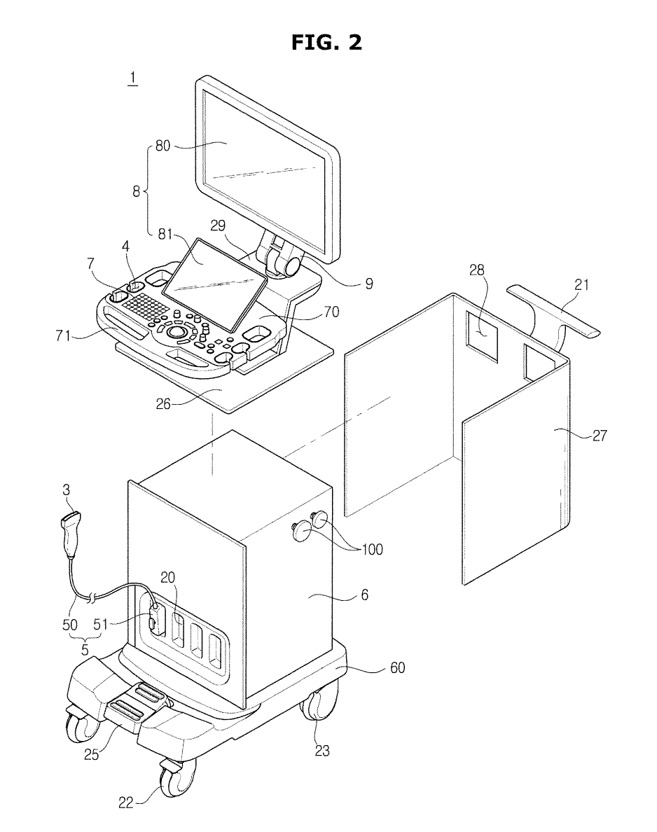

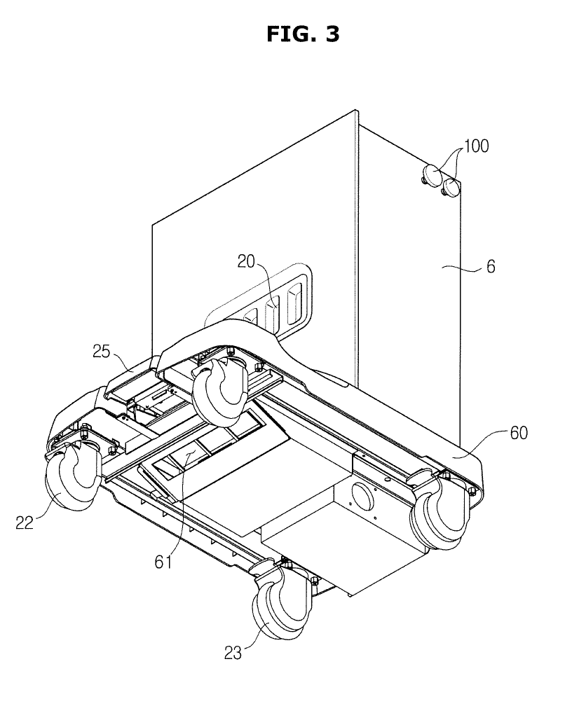

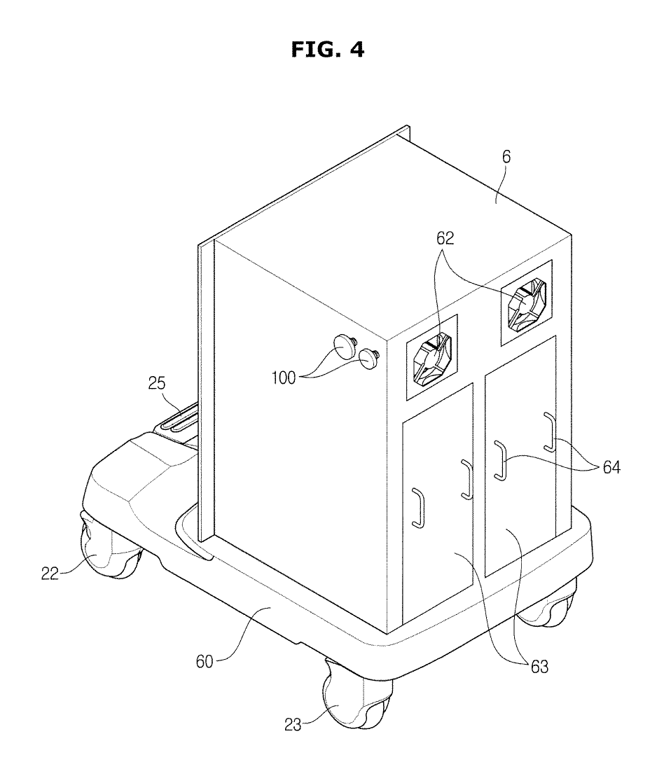

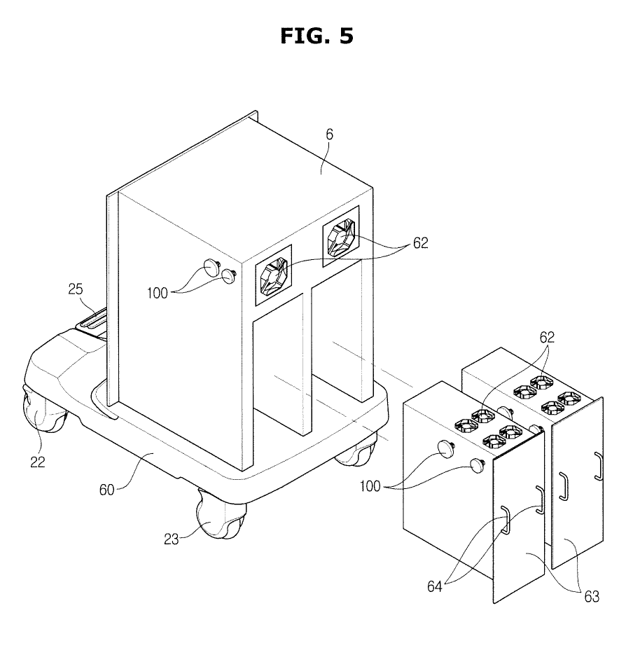

FIG. 2 is an exploded perspective view illustrating the medical imaging apparatus of FIG. 1 with a body separated from a cover, FIG. 3 is a bottom perspective view illustrating the body having the cover shown in FIG. 2 separated therefrom, FIG. 4 is a rear perspective view illustrating the body having the cover shown in FIG. 2 separated therefrom, and FIG. 5 is a perspective view illustrating a processing unit case separated from the body shown in FIG. 4.

Referring to FIG. 2, the body 2 of the ultrasonic imaging apparatus 1 may include a housing 6 accommodating a processing unit, a platform 60 supporting the housing 6 at a lower side of the housing 6, and a cover forming the external appearance of the body 2. In detail, the cover may include a side cover 27 surrounding a left side surface, a right side surface and a rear side surface of the housing 6 and an upper side cover 26 covering an upper surface of the housing 6.

A support part 29 may be connected to the upper surface cover 26 to support the input panel 70 and the display member 8. The second handle part 21 may be connected to the side surface cover 27. In addition, the manipulation part 25 may be provided on the platform 60 to control the casters 22 and 23.

The processing unit may include: a central processing unit (CPU) to process a detection signal transmitted from the detection unit and a control signal input from the control unit; and a heat generating body, such as a circuit element. Accordingly, the housing 6 may include a cooling apparatus to dissipate heat and reduce the internal temperature.

In addition, in order to cool the interior of the body 2, the body 2 may include a suction port 61 allowing outside air to be introduced into the body 2 or the housing 6. The cooling apparatus may be provided as a fan 62 that suctions air outside of the body 2 or the housing 6 or to discharge air inside of the body 2 or the housing 6. The outside air introduced through the suction port 61 may be discharged by the fan 62 after exchanging heat with the processing unit having a high temperature inside the body 2 or the housing 6.

Referring to FIGS. 2 to 4, the fan 62 may be provided at a rear surface of the housing 6, and the suction port 61 may be provide at a lower surface of the platform 60. The suction port 61 and the fan 62 may be appropriately disposed at any other position where cooling is needed. In addition, an exhaust port 28 may be provided at a position of the side surface cover 27 corresponding to the position of the fan 62 disposed on the housing 6.

The cooling apparatus for cooling inside the body 2 or the housing 6 may cause noise as the inner space of the body 2 or the housing 6 serves as a resonance space. In particular, the fan 62 may generate noise by flow of air.

The medical imaging apparatus is used by a patient who is sensitive to sound in a closed space, such as a medical examination room of a hospital, and thus needs to achieve silence. Therefore, the medical imaging apparatus in accordance with an embodiment of the present disclosure may have the noise reducing apparatus disposed inside the body to reduce noise generated from the body.

Referring to FIGS. 2 to 4, a noise reducing apparatus 100 including at least one noise reducing apparatus may be disposed at a side surface of the housing 6. The noise reducing apparatus 100 in accordance with an embodiment of the present disclosure is provided to reduce flow noise mainly caused by the fan 62, and is disposed on a flow path along which air introduced through the suction port 61 moves to the fan 62.

The noise reducing apparatus 100 may be provided as a Helmholtz resonator including a neck and a cavity. Since the Helmholtz resonator has a natural frequency according to the shapes of the neck and the cavity, the noise reducing apparatus may be provided in a different shape depending on the frequency of noise needed to be reduced.

The noise reducing apparatus in accordance with an embodiment of the present disclosure may have the neck 110 and the cavity 120 detachably coupled from each other so that the neck 10 and the cavity 120 are combined with each other in different shapes to form a desired natural frequency.

Arrangement of two or more noise reducing apparatus 100 in different combinations may broaden frequency bands of noise to be reduced. In addition, for medical imaging apparatuses having different frequency band of noise, the neck and the cavity are provided in various combinations, so that the noise reducing apparatus having a desired natural frequency is easily manufactured, and coping with different frequencies of noise among various medical imaging apparatuses in an active manner.

The housing 6 may include a case 63 in which a processing unit is accommodated. The processing unit needs to be mounted to enable easy inspection and replacement for maintenance of the medical imaging apparatus. Accordingly, the plurality of processing units may be accommodated in individual cases 63, respectively, and mounted in the housing 6 or the body 2.

Referring to FIG. 5, the case 63 in accordance with the exemplary embodiment of the present disclosure may be mounted by being inserted into a rear surface of the housing 6. The case 63 may include a knob 64 for the case 63 to be easily inserted into and withdrawn from the housing 6. In addition, the case 63 may separately include the cooling apparatus 62 to dissipate heat generated from the processing unit, and separately include the noise reducing apparatus 100 formed adjacent to the cooling apparatus 62 to reduce flow noise of the cooling apparatus 62.

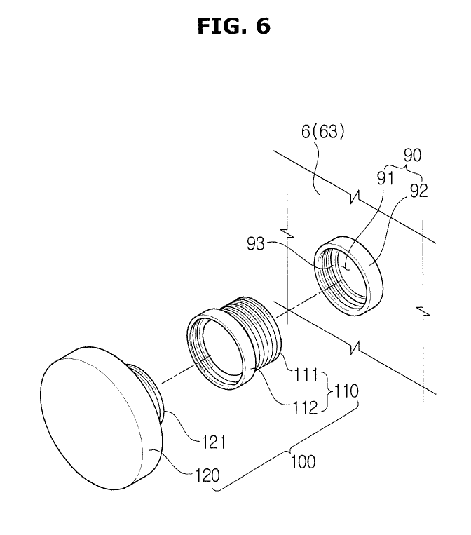

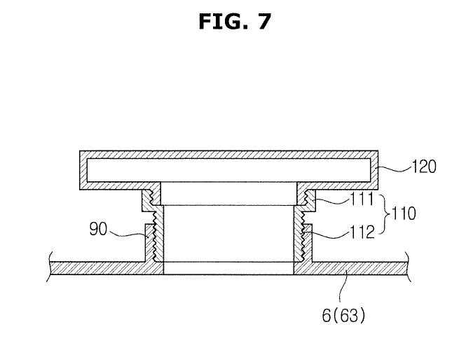

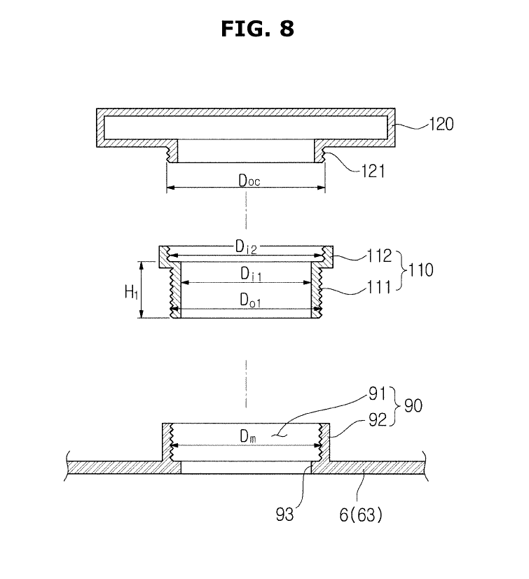

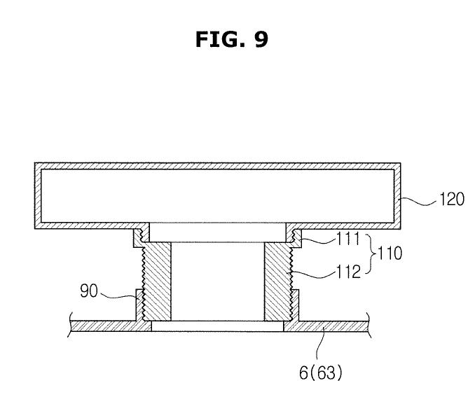

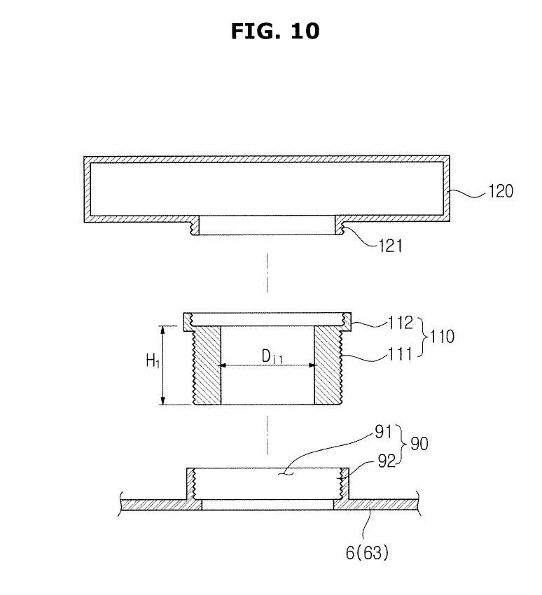

FIG. 6 is an exploded and enlarged perspective illustrating a noise reducing apparatus shown in FIG. 4, FIG. 7 is a cross sectional view illustrating the noise reducing apparatus shown in FIG. 4, FIG. 8 is an exploded cross sectional view illustrating the noise reducing apparatus shown in FIG. 7, FIG. 9 is a cross sectional view illustrating the noise reducing apparatus shown in FIG. 4 in accordance with another embodiment of the present disclosure, and FIG. 10 is a cross sectional view illustrating the noise reducing apparatus of FIG. 9.

Referring to FIG. 6, the noise reducing apparatus 100 in accordance with the embodiment of the present disclosure may be detachably mounted at a mounting part 90 that is provided on one surface of the housing 6 or the case 63. In detail, the neck 110 of the noise reducing apparatus 100 may be detachably coupled to the mounting part 90, and the cavity 120 of the noise reducing apparatus 100 may be detachably coupled to the neck 110.

The neck 110 of the noise reducing apparatus 110 may include a first portion 111 connected to the mounting part 90 and a second portion 112 to which the cavity 120 is connected. The first portion 111 and the second portion 112 may be provided in a hollow cylinder or a hollow square pillar.

The cavity 120 of the noise reducing apparatus 100 is provided in the form of a hollow cylinder or a hollow hexahedral box, and is provided at one side thereof with a connection part 120 coupled to the neck 110 in a shape of a hollow cylinder or a hollow square pillar.

The mounting part 90 may include an opening 91 allowing the noise reducing apparatus 100 to communicate with the inside of the housing 6 or the case 63 and a sidewall connected to the neck 110 of the noise reducing apparatus 100.

Referring to FIGS. 6 to 10, when the neck 110 of the noise reducing apparatus 100 in accordance with an exemplary embodiment of the present disclosure has the first portion 111 and the second portion 112 provided in the form of a cylinder, the connection part 121 of the cavity 120 of the noise reducing apparatus 100 and the sidewall 92 of the mounting part 90 provided on the housing 6 or the case 63 may be provided in the form of a cylinder.

In order for the first portion 111 of the neck 110 of the noise reducing apparatus 100 to be coupled to the mounting part 90, screw threads may be respectively formed on an outer surface of the first portion 111 of the neck 110 and an inner surface of the sidewall 92 of the mounting part 90. In order for the second portion 112 of the neck 110 to be coupled to the connection part 121 of the cavity 120, screw threads may be respectively formed on an inner surface of the second portion 112 of the neck 110 and an outer surface of the connection part 121 of the cavity 120.

In addition, the mount part 90 may include a locking part 93 to prevent the first portion 111 of the neck 110 from being inserted by a predetermined depth or above.

Referring to FIGS. 7 to 10, in order for the neck 110 of the noise reducing apparatus 100 to be replaceable, an inner diameter Dm of the mounting part 90 provided on the housing 6 or the case 63 and an outer diameter Do₁ of the first portion 111 of the neck 110 of the noise reducing apparatus 100 may be provided to be uniform.

Although not shown in the drawing, when the first portion 111 of the neck 110 of the noise reducing apparatus 100 and the mounting part 90 provided on the housing 6 or the case 63 are provided in the form of a square pillar, the size and shape of a cross section the first portion 111 of the neck 110, including an outer wall of the first portion 111, and the size and shape of the opening 91 of the mounting part 90 except for the sidewall 92 may be provided to be uniform.

Referring to FIGS. 7 to 10, in order for the cavity 120 of the noise reducing apparatus 100 to be replaceable, an inner diameter Di₂ of the second portion 112 of the neck 110 and an outer diameter Doc of the connection part 121 of the cavity 120 may be provided to be uniform.

Although not shown in the drawing, when the second portion 112 of the neck 110 of the noise reducing apparatus 100 and the connection part 121 of the cavity 120 are provided in the form of a square pillar, the size and shape of a cross section of a hollowness of the second portion 112 of the neck except for an outer wall of the second portion 112 and the size and shape of a cross section of the connection part 121 of the cavity 120 including an outer wall of the connection part 121 may be provided to be uniform.

In addition, although not shown in the drawing, the neck 110 of the noise reducing apparatus 100 and the mounting part 90 may be integrally formed with each other such that only the cavity 120 of the noise reducing apparatus 100 is replaceable according to frequencies of noise generated from the medical imaging apparatus. Alternatively, the neck 110 and the cavity 120 of the noise reducing apparatus 100 may be integrally formed with each other such that the noise reducing apparatus is replaceable according to frequencies of noise generated from the medical imaging apparatus.

Referring to FIGS. 7 to 10, in order for the neck 110 and the cavity 120 of the noise reducing apparatus 100 to be combined with each other according to frequencies of noise generated from the medical imaging apparatus, the inner diameter Di₁ of the first portion 111 or the cross section of the hollowness of the first portion 111 except for the outer wall of the first portion 111 may be provided in various sizes, and also, a height H₁ of the first portion 111 may be provided in various sizes. In addition, the cavity 120 may be provided in various inner volumes.

If a plurality of frequency bands of nose are generated from the medical imaging apparatus, the noise reducing apparatus 100 may include a plurality of necks 110 and a plurality of cavities 120, and the housing 6 or the case 63 may include a plurality of mounting parts 90. In this case, the sizes and shapes of the inner diameters Dm of the plurality of mounting parts 90 or the openings 91 of the plurality of mounting parts 90 except for the sidewalls 92 of the plurality of mounting parts 90 may be provided to be uniform.

As is apparent from the above, noise generated from the medical imaging apparatus can be reduced in an active manner by disposing a noise reducing apparatus including a neck and a cavity that are properly combined with each other depending on frequency of noise generated from the medical imaging apparatus, thereby achieving improved silence.

Although a few embodiments of the present disclosure have been shown and described, it would be appreciated by those skilled in the art that changes may be made in these embodiments without departing from the principles and spirit of the disclosure, the scope of which is defined in the claims and their equivalents.

What is claimed is:

1. A medical imaging apparatus comprising:
a body and
a noise reducing apparatus disposed inside the body to reduce noise generated from the body,
wherein the noise reducing apparatus includes a neck and a cavity, and
the neck is combined with the cavity according to a frequency of the noise desired to be reduced.

2. The medical imaging apparatus of claim 1, wherein
the body further includes a cooling apparatus to cool heat inside of the body, and
the noise reducing apparatus reduces noise generated from the cooling apparatus.

3. The medical imaging apparatus of claim 1, further comprising:
a processing unit disposed inside the body to process a medical image; and
a housing disposed inside the body to accommodate the processing unit,
wherein the noise reducing apparatus is mounted at the housing.

4. The medical imaging apparatus of claim 3, wherein the housing includes a mounting part on which the noise reducing apparatus is mounted, and
the neck of the noise reducing apparatus is detachably coupled to the mounting part and the cavity is detachably coupled to the neck.

5. The medical imaging apparatus of claim 4, wherein the neck includes a first portion connected to the mounting part and a second portion to which the cavity is connected, and
the first portion or the second portion is provided in the form of a hollow cylinder or a hollow square pillar.

6. The medical imaging apparatus of claim 5, wherein the cavity is provided in the form of a cylinder or a hexahedral box that has an inside thereof empty, and the cavity is provided at one side thereof with a connection part connected to the neck in the form of a hollow cylinder or a hollow square pillar.

7. The medical imaging apparatus of claim 6, wherein the neck includes a screw thread formed on an outer surface of the first portion so as to be coupled to the mounting part and a screw thread formed on an inner surface of the second portion so as to be coupled to the cavity, and
the cavity includes a screw thread formed on an outer surface of the connection part so as to be coupled to the second portion of the neck.

8. The medical imaging apparatus of claim 6, wherein each of the neck and the cavity of the noise reducing apparatus is provided in plural,
outer diameters of the first portions of the plurality of necks or cross-sections of the first portions including outer walls of the first portions of the plurality of necks have uniform sizes and shapes among each other, and
inner diameters of the first portions of the plurality of necks or cross-sections of hollow portions of the plurality of necks except for outer walls of the first portions of the plurality of necks have different sizes and shapes from each other.

9. The medical imaging apparatus of claim 8, wherein
outer diameters of the connection parts of the plurality of cavities or cross-sections of the connection parts of the plurality of cavities including outer walls of the connection parts of the plurality of cavities have uniform sizes and shapes among each other, and
inner volumes of the plurality of cavities are different from each other.

10. The medical imaging apparatus of claim 4, wherein the mounting part is formed on one surface of the housing,
wherein the mounting part includes:
an opening allowing the inside of the housing to communicate with the noise reducing apparatus and
a sidewall surrounding the opening and to which the neck of the noise reducing apparatus is connected.

11. The medical imaging apparatus of claim 10, wherein a screw thread is formed on an inner surface of the sidewall of the mounting part so as to be coupled to the neck.

12. The medical imaging apparatus of claim 3, wherein the housing includes a cooling apparatus to cool heat inside of the housing generated from the processing unit, and
the noise reducing apparatus reduces noise generated from the cooling apparatus.

13. The medical imaging apparatus of claim 12, wherein the body further includes:
a platform configured to support the housing at a lower side of the housing; and
a cover configured to encase the housing to form an external appearance of the body,
wherein the platform or the cover is provided with a suction portion allowing outside air to be introduced into the body.

14. The medical imaging apparatus of claim 13, wherein the cooling apparatus is provided as a fan that discharges air suctioned through the suction port, and
the noise reducing apparatus is disposed on a flow path along which air introduced through the suction port moves to the fan.

15. The medical imaging apparatus of claim 13, wherein the cooling apparatus is mounted on a rear surface of the housing, and
the noise reducing apparatus is mounted on a upper surface or a side surface of the housing.

16. The medical imaging apparatus of claim 15, wherein the cover includes an exhaust port provided at a position corresponding to the cooling apparatus that is disposed on the rear surface of the housing.

17. The medical imaging apparatus of clam 3, wherein the processing unit is accommodated in a case,
the case includes a separate fan and a separate noise reducing apparatus, and
the case is disposed inside the housing.

18. A medical imaging apparatus comprising:
a detection unit configured to acquire a detection signal and transmit the acquired detection signal;
a body including a processing unit that is configured to convert the detection signal transmitted from the detection unit into an image signal; and
an output unit configured to receive the image signal from the processing unit and display the received image signal,
wherein the body includes a noise reducing apparatus configured to reduce noise generated from the body.

19. The medical imaging apparatus of claim 18, wherein the body further includes a cooling apparatus to cool heat inside of the body, and
the noise reducing apparatus reduces noise generated from the cooling apparatus

20. The medical imaging apparatus of claim 18, wherein the noise reducing apparatus is detachably mounted inside of the body so as to be replaced according to a frequency of noise desired to be reduced.

21. The medical imaging apparatus of claim 18, wherein the noise reducing apparatus includes a neck and a cavity,
wherein the neck is detachably coupled to the body and the cavity is detachably coupled to the neck, so that the neck is combined with the cavity according to a frequency of the noise desired to be reduced.

22. The medical imaging apparatus of claim 19, wherein the body includes a suction portion allowing outside air to be introduced into the body, and
the noise reducing apparatus is disposed on a flow path along which air introduced through the suction port moves to the cooling apparatus.
